(19) United States
(12) Patent Application Publication
(10) Pub. No.: US 2018/0208302 A1
(43) Pub. Date: Jul. 26, 2018

(54) COMPOSITE COMPONENTS MADE OF HEAT-CURING RESINS AND ELASTOMERS

(71) Applicant: Gummiwerk Kraiburg GMBH & Co. KG, Waldkriaburg (DE)

(72) Inventors: Jens Schaube, Ampfing (DE); Judith Zahn, Traunreut (DE); Florian Plenk, Muehldorf (DE)

(21) Appl. No.: 15/918,225

(22) Filed: Mar. 12, 2018

Related U.S. Application Data
(63) Continuation of application No. 13/263,855, filed on Oct. 11, 2011, now abandoned.

(30) Foreign Application Priority Data
Apr. 14, 2009 (DE) 20 2009 005 438.1
May 14, 2009 (DE) 20 2009 006 966.4

(51) Int. Cl. B64C 27/473; A63C 11/22; B29C 70/08; B32B 17/04; B32B 25/08; B32B 27/04; B60R 25/04; B60R 25/10; F03D 1/06; B63H 16/04; B63B 35/79; B63B 35/71; B63B 5/24; B63B 3/68; A63C 5/12; A61F 2/50

(52) U.S. Cl. CPC B64C 27/473; Y02E 10/721; B29C 70/086; B32B 17/04; B32B 25/08; B32B 27/04; B60R 25/04; B60R 25/10; F03D 1/065; B63H 16/04; B63B 35/7909; B63B 35/71; B63B 5/24; B63B 3/68; B60R 2325/304; A63C 5/122; A61F 2002/5056; A61F 2002/5055; Y10T 442/2926; Y10T 428/31855; Y10T 428/31826; Y10T 428/31663; Y10T 428/31504; B64C 2027/4736; F05B 2260/96; F05B 2280/5001; F05B 2280/6003; F05C 2251/02; F05C 2253/04; Y02T 50/43; Y02T 70/143; A63C 11/227

(57) ABSTRACT

A plastic composite component which is formed by a thin hard plastic outer layer, at least one elastomer layer adjoining the former on the inside, and at least one metal and/or plastic carrier layer adjoining said elastomer layer on the inside and made of a fibre reinforced plastic (RFP). A carbon fibre reinforced plastic (CRP) or glass fibre reinforced plastic (GRP), is arranged on the component or at least partly forms the latter, as an impact protection part, as a splinter protection part, as a protective part against sudden total failure or as a protective part against vibrations and vibration damages, against resonance, for the purpose of damping oscillations or for the purpose of acoustic damping.

### CROSS-REFERENCE TO RELATED APPLICATIONS

This application is a Continuation of application Ser. No. 13/263,855, filed on Oct. 11, 2011, which is the U.S. National Phase of PCT Application No. PCT/EP2010/002293, filed on Apr. 14, 2010, which claims priority to German application 20 2009 005 438.1 filed Apr. 14, 2009 and German application 20 2009 006 966.4 filed May 14, 2009, the disclosures of which are incorporated herein by reference.

### FIELD OF THE INVENTION

The invention broadly relates to composite components made of heat-curing resins and elastomers according to the preamble of the independent claims.

### BACKGROUND OF THE INVENTION

The current background for producing composite components from a heat-curing resin with an elastomer layer consists in first producing a molding from heat-curing resin and applying the elastomer layer thereto in a new processing step. This system is currently used in the case of elastomer-coated rollers or cylinders as well as other multi-component moldings. The production and curing of the resin-based molding take place in autoclaves or hot presses at elevated temperature, wherein a reinforcing insert made of a woven fabric or fibrous materials can additionally be incorporated. In both processes the curing occurs by chemical reaction. For example, rollers, wheels, anti-slip coatings made of plastics material or elastomer articles provided with a certain inherent rigidity can be produced in this manner.

With use of plastic plates, for example in automotive construction or shipbuilding, elastomer profiled parts are fixed, for example glued on or screwed on, to the edges so as to create a seal and compensate for different coefficients of thermal expansion or so as to avoid warping and creaking in the event of resilient movements of the vehicles caused by travelling conditions.

Fiber-reinforced plastics are generally energy elastic and brittle and can therefore take on or absorb little energy in the event of an application of energy caused by vibrations, collisions, impacts or bombardment. This may lead to damage of the component, wherein sharp and jagged breaking edges occur which may cause injury. If necessary, specific measures must be taken to absorb energy and prevent damage to the component caused by resonances. Fiber-reinforced plastics are easily combustible owing to their resin content and, in the event of a fire, supply additional fuel thereto.

In WO 2006/122749 A1 the applicant has already described composite components, methods for the production thereof and some advantageous applications.

The object of the present invention is to describe further advantageously producible plastic composite components which are of increased customer benefit in different fields.

This object is achieved by the plastic composite components disclosed in the different independent claims. Advantageous embodiments of the invention are to be inferred from the respective dependent claims.

In accordance with the invention a hard layer is formed as a component surface from the heat-curing resin by chemical reaction, at the same time the elastomer layer provided with a cross-linking agent is also cured or vulcanized by chemical reaction, the direct bond between the elastomer layer and the hard layer is achieved, and lastly the connection to a plastics carrier layer arranged on the rear of the elastomer layer is formed.

A woven fabric insert, a fiber material or metal powder may be contained in one or more layers.

The terms “plastics outer layer” and “adjoining on the inside” are each considered from the side of use of the plastic composite component. In the case of an inner cladding part of a vehicle, the plastics outer layer is accordingly the layer facing the interior of the vehicle.

If the present invention refers to “layers”, this may mean strips, pieces or areas of the aforementioned materials arranged above one another or embedded in one another either completely or only in part.

Within the meaning of the present invention a plastic composite component is also referred to if one or more layers of the plastic composite component consist of metal or other materials not to be referred to as plastics.

The plastics carrier layer acting as a solid carrier and which can also optionally be supplemented or replaced by at least one metal carrier layer, the elastomer layer located thereon and the plastics outer layer are jointly assembled in a single processing step and are then cured or vulcanized jointly under the influence of heat in an autoclave or a hot press. All raw materials involved are adapted to one another in such a way that they simultaneously form a chemical network under identical reaction conditions and form a bond to one another. A dimensionally stable product is produced by these processes. The curing temperature is preferably between 80 and 200° C.

The assembly of the multi-component product made of heat-curing resin, the elastomer layer provided with a cross-linking agent and the metal and/or plastics carrier layer occurs in a number of alternatives as follows:

in accordance with a first alternative an anti-adhesively coated mold is loaded with the different raw components of the individual layers and the composite part is cured under the influence of pressure and heat (hot-pressing);

in accordance with a second alternative the product prefabricated from the raw materials is cured without the use of a closed mold at elevated temperature in an autoclave or a hot-air oven. It may be fixed on a carrier member forming the plastics carrier layer;

in accordance with a third alternative the product prefabricated from the raw materials is cured without the use of a closed mold at elevated temperature in a vacuum bag.

The anti-adhesive coating of the mold may be produced by paraffins, silicone, surfactants or fluorocarbons (for example Teflon).

The following may preferably be used as synthetic resins: polyester resins, phenol formaldehyde resins, cyanate ester resin, epoxy resins and acrylate resins.

The elastomer components which are not yet cross-linked but are provided with a cross-linking agent and the woven fabric inserts are laid directly in the mold at the corresponding locations during production of the fiber-reinforced plastic parts. The following may preferably be used as an insert in the composite product: glass fibers, nylon, polyester, carbon fibers, viscose, aramid fibers and/or metal fibers. The insert may be provided in the form of a woven fabric, a non-woven fabric or a pulp.

With use of a thermoplastic elastomer (TPE) for the elastomer layer in conjunction with a thermosetting plastics carrier layer, the TPE is optionally heated before being combined with the thermoset to a temperature in the vicinity of the softening point of the TPE. The TPE can thus be better draped over the thermoset. It can adapt better to the contour thereof and/or to the contour of a mold or die which is used for the production of the plastic composite component.

By introducing soft elastomer layers both a vibration absorption and a vibration insulation can be achieved by the selection of suitable materials. The spreading of cracks is inhibited, wherein the spreading of cracks is prevented in particular by a woven fabric, knitted fabric or a fiber structure made of high-performance fibers (such as aramid or Vectran®, registered mark of Kuraray Co., Ltd., JP) embedded completely in an elastomer material.

The spread of fire is impaired by the optional introduction of flame-resistant elastomer layers.

The production of an electrically conductive or radiation-screening variation is possible without significantly higher material costs caused by the embedding of conductive fibers, strips or woven fabric.

A particularly advantageous property of a plastic composite component according to the invention consists in that the plastic composite component absorbs energy and therefore can be used in all areas in which components have to be protected against mechanical energies and impulses or, vice versa, in which people or objects have to be protected against impacting composite components.

A further advantageous property of a composite component according to the invention consists in that the composite component exhibits improved crash behavior with the consequence of effective splinter protection to avoid injury and an avoidance of a sudden total failure of the composite component.

In the generic document WO 2006/122749 A1 already mentioned above, a wide range of advantageous applications for a plastic composite component according to the invention are already disclosed, but with no detailed indication of the layered structure which is particularly advantageous for the respective application or of the preferred field of application:

rotor blade, for example of a wind wheel or helicopter

airfoil of aircraft

leaf spring, for example in automotive construction

impact- or missile-resistant component in protective clothing, such as protectors in motorbike clothing or bullet-proof vests or armor-plated vehicles

stable vibration-damping core of sports equipment, such as skis, snowboards, sleds, bobsleighs, surfboards, boats, tennis rackets or ice hockey sticks

bearings for machines, machine parts, bridges and other structures and structure parts

housing for valuable consumer goods, such as computers or notebooks

walling of pipes, elastomer-coated cylinders or pipes

cladding or bodywork part of vehicles, as a cylinder head cover of engines or as a bumper

components with reduced tendency for breaking or splintering with deformation

flame-resistant composite parts by installation of incombustible resilient layers (for example metal or metal hydroxide particles or halogenated paraffins as aggregates in the elastomer)

self-destructive and fire-resistant rubber mixtures

The generic document WO 2006/122749 A1 further mentions that a multi-layer structure is advantageous for specific applications, wherein a synthetic resin layer and an elastomer layer provided with a cross-linking agent advantageously alternate and wherein one or both outer layers are advantageously formed by synthetic resin. However, the sandwich construction may also be reversed if soft outer layers are desired, for example in the case of a table tennis bat, an inner cladding part of a vehicle or a mouse mat.

The advantages disclosed in the generic document WO 2006/122749 A1 also apply, in principle, to the plastic composite components described in the present application:

the separate production of a resin-based solid carrier is no longer necessary;

the entire molding is formed in a continuous processing step at the same workstation;

a use of adhesion promoters is not necessary;

lower expenditure of time for production and therefore considerable cost reduction.

In principle it is also advantageous for the plastic composite components described in the present application if, as described in the generic document WO 2006/122749 A1, the elastomer layers contain at least 0.5 pph (parts per hundred) of at least one cross-linking agent from the group of peroxides, amines and/or bisphenols, and both the carrier layer and the elastomer layer can be interconnected by the influence of heat or another form of energy application in a single processing step, without the need for an adhesion promoter. Such cross-linking agents are not necessary with use of a thermoplastic elastomer (TPE), such as styrene/ethylene butene/styrene block copolymer (SEBS) or styrene/butadiene/styrene block copolymer (SBS) or a thermoplastic elastomer based on polyurethane (TPU) or a low-density polyethylene (LDPE) or styrene/butadiene rubber (SBR) with a styrene content of more than 50%.

### BRIEF SUMMARY OF THE INVENTION

The present invention widens the applications already proposed since at least one thin hard plastics outer layer made of synthetic resin and an elastomer layer adjoining the former are jointly connected using a metal and/or plastics carrier layer to form a plastic composite component, wherein the plastics carrier layer is formed of a fiber-reinforced plastic (FRP), a carbon fiber reinforced plastic (CRP) or a glass fiber reinforced plastic (GFP). Such an alternating “hard-soft-hard” layer structure has proven to be particularly advantageous for the plastic composite components disclosed in independent claims 1 to 3, wherein the key aspects of the three independent claims are three essentially different applications:

impact protection according to claim 1;

improved crash behavior (splinter protection, avoidance of total failure of a component) according to claim 2, and;

improved behavior in relation to vibrations according to claim 3.

Of these three applications, at least two may overlap in some components. For example, in the case of a bumper or a bonnet of a vehicle, not only the active and passive safety of the passengers and any individuals colliding with the vehicle is important, but also a splinter-free deformation for absorbing the impact energy and a reduction in the impulse upon impact of small bodies (stone-chipping). However, bonnets also should not generate any droning noises caused by vibrations of the vehicle or the drive thereof, and therefore both the first two aspects and also the third aspect of vibration damping are important in this instance.

In a fourth application according to claim 33 the plastic composite component is formed of at least one thin hard plastics outer layer made of synthetic resin and an elastomer layer adjoining the former with a woven fabric, knitted fabric or fiber layer embedded therein. In this case the plastic composite component has bi-flexible properties, wherein the deflection in the event of an application of force from the elastomer side is stronger than in the event of application of an identical force from the synthetic resin side. In other words, in the event of an application of force from the synthetic resin side, the plastic composite component has a higher modulus of elasticity than in the event of an application of force from the elastomer side. This property opens up completely new applications in many technical fields. Examples include components loaded aerodynamically or hydrodynamically from different sides under different operating conditions, such as spoilers, airfoils, tail units, and valve membranes, which are exposed to compressive forces from one side and base drag forces from the other side and which, owing to their different deflection, control or assist specific functions (lift, downforce, leakiness in valves) merely by their material properties. Such a plastic composite component is also suitable for use as a hinge which can be bent preferably in one direction (specifically the bending direction with the lower modulus of elasticity).

A fifth field of application for a plastic composite component according to claim 34 made of at least one thin hard plastics outer layer made of synthetic resin, at least one elastomer layer and at least one woven fabric, knitted fabric or fiber layer is a flexible design provided in a defined area (articulation region) of the plastic composite component, which design enables a hinge-like movability of the adjacent areas of the plastic composite component. The flexible area of the plastic composite component is characterized in that at least one elastomer layer, optionally more elastomer layers and also optionally at least one woven fabric, knitted fabric or fiber layer are provided in said plastic composite component, whereas the other layers of the plastic composite component, in particular the synthetic resin layers are preferably omitted in this area. This fifth field of application also enables a large number of new applications in different technical fields. Examples include:

door or flap hinges, for example for buildings, furniture or vehicles, but also for suitcases, chests, containers or other receptacles, wherein the tight design of the hinge area continuously along the hinge edge prevents the infiltration of air, liquid or particles in this area;

seals which have to be adapted to three-dimensional contours;

kinked areas of plates or hoses;

compensators for compensation of a vertical or lateral offset between two component faces;

building claddings in corner regions (“soft edge protection” for example on pillars or quoins of underground parking areas);

articulation of movable flaps, such as landing flaps of aircraft, flow-directing flaps at retaining dams, rotor blades of helicopters;

flexible suspensions (for example in motor sport).

Depending on the layer structure with which the articulation region of the plastic composite component is formed, the articulation obtains one or more degrees of freedom:

only kinks or bends by at least one layer of synthetic resin (prepreg) which is preferably embedded in one or between two elastomer layers;

kinks and torsion by at least one layer of a bi-directional woven fabric;

kinks, torsion and diagonal displacement by at least one layer of a uni-directional woven fabric or directed fibers;

all degrees of freedom by at least one layer of an elastomer (for example rubber or silicone).

In the aforementioned list the movability of the articulation increases from top to bottom. The lower layers thus do not form any limitation for layers above and can therefore be used in conjunction therewith.

In an embodiment which is advantageous for all applications, the plastics outer layer is formed of a woven fabric material which is already saturated with synthetic resin (prepreg). Alternatively, the plastics outer layer is formed or produced by means of the resin infusion method.

Alternatively, dry fibers (woven fabrics, non-woven fabrics or pulps) can be laid in an elastomer layer and then connect to the elastomer layer and optionally also to adjacent layers. A dry fiber layer embedded in the elastomer layer or TPE layer acts similarly to a film in a laminate glass panel: if the composite component breaks, it holds all the individual parts thereof together.

The plastics outer layer is preferably formed of a fiber-reinforced composite plastic (FRP, CRP, GRP), polyethylene (PE), in particular a high molecular weight polyethylene (HMW-PE, or ultra high molecular weight polyethylene—UHMW-PE) or polytetrafluoroethylene (PTFE). The surface of the plastics outer layer is therefore relatively hard and preferably also very smooth. The plastics outer layer and/or the plastics carrier layer may alternatively also be formed by an “organic sheet” or a thermoplastic polymer with an embedded long-fiber reinforcement or endless-fiber reinforcement. In this context, a fiber length of 2 mm to 50 mm refers to long fibers; endless fibers are understood by a person skilled in the art to be fibers with a fiber length over 50 mm (see DE 10 2007 036 660 A1). In particular, with use of an organic sheet as a plastics outer layer and/or as a plastics carrier layer the plastic composite component can be produced together with its other layers in a single processing step by pressing or thermoforming.

At least one woven fabric or a knitted fabric or a fiber structure is preferably embedded in the elastomer layer in such a way that the fibers thereof are surrounded completely by the elastomer, at least in the partial areas subject to particular stress. The embedding within the elastomer layer is produced so that the woven fabric or the knitted fabric or the fiber structure is arranged closer to the side of a tensile load or bending tensile load of the plastic composite component. The woven fabric material of the carrier layer preferably consists of glass fibers, nylon, polyester, carbon fibers, viscose, aramid fibers or metal fibers. The fibers may be arranged in the form of a woven fabric, a non-woven fabric or a pulp. Polyester resin, phenol formaldehyde resin, cyanate ester resin, epoxy resin or acrylate resin can particularly preferably be used as synthetic resin. In particular, the woven fabric or the knitted fabric or the fiber structure in such plastic composite components in which a splintering and sudden total component failure are to be avoided at all costs particularly preferably consist of a high-performance fiber such as aramid or Vectran® (registered mark of Kuraray Co., Ltd., JP).

Provided it does not consist of TPE, the elastomer layer contains a cross-linking system which, depending on the elastomer used, contains at least one cross-linking agent from the group of peroxides, amines and/or bisphenols and enables a reaction with the synthetic resin of the carrier layer. Alternatively to a heat treatment for a cross-linking of the elastomer layer with the synthetic resin of the carrier layer, another cross-linking treatment, for example with ultraviolet radiation (UV light) may also take place. Further elastomer layers, if necessary with different strength and hardness, can be applied to a first elastomer layer and are composed in such a way that they bond to the respective elastomer layer located therebeneath. The at least one elastomer layer particularly preferably consists of materials based on rubber. Alternatively, the at least one elastomer layer may also consist of a thermoplastic elastomer (TPE).

These and other objects and advantages of the present invention will be readily appreciable from the following description of preferred embodiments of the invention and from the accompanying drawings and claims.

### BRIEF DESCRIPTION OF THE DRAWINGS

The nature and mode of operation of the present invention will now be more fully described in the following detailed description of the invention taken with the accompanying drawing figures, in which:

FIG. 1 is a schematic view of the basic layer structure of a plastic composite component.

FIG. 2 is a plan view of a rotor blade.

FIG. 3 is a sectional view through the front edge region (in the direction of rotation) of the rotor blade.

FIG. 4 is a sectional view through a plastic composite component acting as an edge protection part or a splinter protection part.

FIG. 5 is a plan view of a bicycle handlebar.

FIG. 6 is a sectional view through the central region of the bicycle handlebar according to FIG. 5.

FIG. 7 is a plan view of a planar plastic composite component with strip-like vibration-damping regions.

FIG. 8 shows a section through a strip-like vibration-damping region according to FIG. 7 in a first variation.

FIG. 9 shows a section through a strip-like vibration-damping region according to FIG. 7 in a second variation.

FIG. 10 shows a section through a plastic composite component with planar vibration damping in a first variation.

FIG. 11 shows a section through a plastic composite component with planar vibration damping in a second variation.

FIG. 12 shows a section through a plastic composite component with planar vibration damping in a third variation.

FIG. 13 shows a section through a plastic composite component with planar vibration damping in a fourth variation.

FIG. 14 shows a section through a plastic composite component with planar vibration damping in an idle position.

FIG. 15 shows a section through a plastic composite component with planar vibration damping in a position deflected by vibrations.

FIG. 16 shows a section through a vibration-damping plastic composite component.

FIG. 17 shows a section through a bi-resilient plastic composite component in the unloaded state.

FIG. 18 shows the plastic composite component according to FIG. 17 with loading from the hard layer side.

FIG. 19 shows the plastic composite component according to FIG. 17 with loading from the soft layer side.

FIG. 20 shows a plastic composite component with a hinge-like movable connection region (articulation region).

FIG. 21 shows a plastic composite component with a core layer made of an elastomer.

### DETAILED DESCRIPTION OF THE INVENTION

At the outset, it should be appreciated that like drawing numbers on different drawing views identify identical, or functionally similar, structural elements of the invention. While the present invention is described with respect to what is presently considered to be the preferred aspects, it is to be understood that the invention as claimed is not limited to the disclosed aspects.

Furthermore, it is understood that this invention is not limited to the particular methodology, materials and modifications described and as such may, of course, vary. It is also understood that the terminology used herein is for the purpose of describing particular aspects only, and is not intended to limit the scope of the present invention, which is limited only by the appended claims.

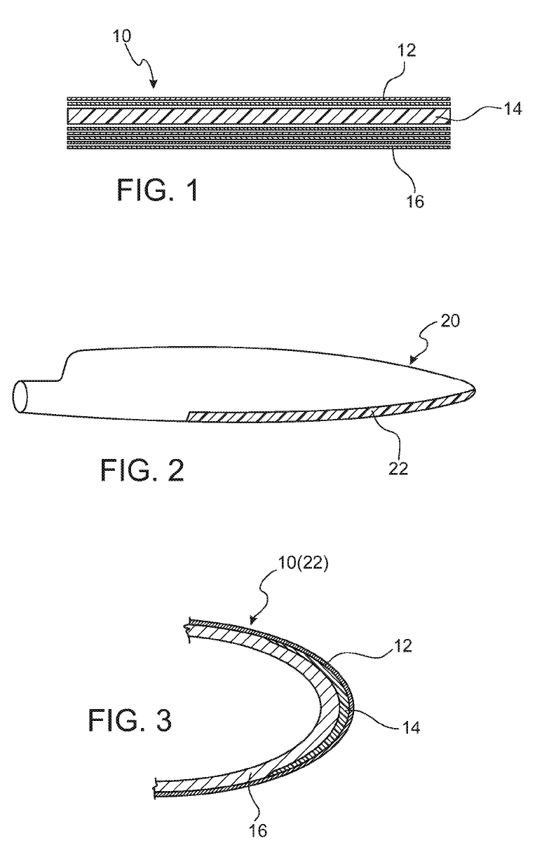

The plastic composite component 10 illustrated in FIG. 1 consists of a plastics outer layer 12, an elastomer layer 14 adjoining the former on the inside and a plastics carrier layer 16 adjoining said elastomer layer on the inside.

The plastics outer layer 12 consists of one or two fiber layers which are saturated with liquid synthetic resin. The fiber layers of the plastics outer layer 12 saturated with synthetic resin may be formed as a prefabricated component in the form of a fiber mat saturated with synthetic resin (prepreg) or may be produced by the resin infusion method. The plastics outer layer 12 is preferably formed of a fiber-reinforced composite plastic (FRP, CRP, GRP) or polyethylene (PE), in particular a high-density polyethylene (HMW-PE—high molecular weight polyethylene or UHMW-PE—ultra high molecular weight polyethylene).

The elastomer layer 14 consists of one of the following substances: ethylene-propylene rubber (EPM), ethylene-propylene-diene rubber (EPDM), ethylene-acrylate rubber (EAM), fluorocarbon rubber (FCM), acrylate rubber (ACM), acrylonitrile-butadiene rubber (NBR), optionally mixed with polyvinyl chloride (PVC), hydrogenated nitrile rubber (HNBR), carboxylate-nitrile rubber (XNBR), hydrogenated carboxylate-nitrile rubber (XHNBR), natural rubber (NR), ethyl vinyl acetate (EVA), chlorosulfonyl-polyethylene rubber (CSM), chlorinated polyethylene (CM), butyl or halobutyl rubber, silicone rubber (VMQ, MVQ), fluorosilicone rubber (FVMQ, MFQ), chlorohydrin rubber (CO), epichlorohydrin rubber (ECO), polychloroprene rubber (CR), one-component polyurethane (PU) or a combination or a blend of the aforementioned substances. Alternatively, the at least one elastomer layer may consist of a thermoplastic elastomer (TPE).

Provided it doesn't consist of a thermoplastic elastomer (TPE), the elastomer layer 14 contains a cross-linking system which enables a reaction with the synthetic resin of the outer layer 12 and the plastics carrier layer 16. Depending on the elastomer used for the elastomer layer 14, the following materials from at least one of the groups of peroxides, amines and/or bisphenols are suitable as cross-linking agents:

| | Peroxide | | |
|---|---|---|---|
| Elastomer | (yes/no or coagent) | Amine | Bisphenol |
| Ethylene- | Methacrylate | no | no |
| propylene rubber | Acrylate | | |
| (EPM); | Phenol resin | | |
| Ethylene- | Hexamethyl-enetetramine | | |
| propylene-diene | (HMTA) | | |
| rubber (EPDM) | Hexamethoxymethyl- | | |
| | melamine (HMMM) | | |
| Ethylene-acrylate | yes | yes | no |
| rubber (EAM) | | | |
| Fluorocarbon | yes | yes | yes |
| rubber (FCM) | | | |
| Acrylate rubber | yes | yes | no |
| (ACM) | | | |
| Acrylonitrile- | Methacrylate | no | no |
| butadiene rubber | Acrylate | | |
| (NBR), optionally | Phenol resin | | |
| mixed with | Hexamethyl-enetetramine | | |
| polyvinyl chloride | (HMTA) | | |
| (PVC); | Hexamethoxymethyl- | | |
| Hydrogenated | melamine (HMMM) | | |
| nitrile rubber | | | |
| (HNBR); | | | |
| Hydrogenated | | | |
| carboxylate-nitrile | | | |
| rubber (XHNBR) | | | |
| Carboxylate- | Peroxide | no | no |
| nitrile rubber | Zinc peroxide | | |
| (X-NBR) | | | |
| Natural rubber | Methacrylate | no | no |
| (NR) | Acrylate | | |
| | Phenol resin | | |
| | Hexamethyl-enetetramine | | |
| | (HMTA) | | |
| | Hexamethoxymethyl- | | |
| | melamine (HMMM) | | |
| Ethyl vinyl | Methacrylate | yes | no |
| acetate (EVA); | Acrylate | | |
| Chloro-sulfonyl- | Phenol resin | | |
| polyethylene | Hexamethyl-enetetramine | | |
| rubber (CSM); | (HMTA) | | |
| Chlorinated | Hexamethoxymethyl- | | |
| polyethylene | melamine (HMMM) | | |
| (CM), for | | | |
| example Tyrin ® | | | |
| Butyl rubber | Bismaleimide | no | no |
| (BIIR); | m-Phenylene-bismaleimide | | |
| Halobutyl rubber | (HVA-2) | | |
| Silicone rubber | yes (acrylates) | no | no |
| (VMQ, MVQ) | | | |
| Fluoro-silicone | yes (acrylates) | no | no |
| rubber (MFQ, | | | |
| FVMQ) | | | |
| Polyurethane | yes (acrylates) | no | no |
| (PU, | | | |
| one-component) | | | |
| Chlorohydrin | yes | Thiourea and | no |
| rubber (CO) | | derivatives | |
| Epichloro-hydrin | | inter alia | |
| rubber (ECO) | | | |
| Polychloro-prene | yes | Thiourea and | no |
| rubber (CR) | | derivatives | |
| | | inter alia | |

The proportion of cross-linking agent or cross-linking agents in the elastomer material is approximately between 0.5 to 15 pph rubber (parts per 100 parts of rubber of the rubber mixture), but can also be considerably higher.

The plastics carrier layer 16 is preferably formed of at least one layer of a fiber-reinforced plastic (FRP), a carbon fiber reinforced plastic (CRP) or a glass fiber reinforced plastic (GRP). Alternatively or in addition, at least one layer of the plastics carrier layer 16 may also consist of another material, in particular of metal. As a further alternative, the plastics outer layer and/or the plastics carrier layer may also be formed of an “organic sheet”, or a thermoplastic polymer with an embedded long-fiber reinforcement or endless-fiber reinforcement.

In the embodiment shown by way of example in FIGS. 2 and 3 the plastic composite component is used for protection against impacting objects (impact protection). For example, a rotor blade 20 of a wind wheel is illustrated in FIG. 2, in which a plastic composite component 22 is arranged at least in the region of the edge arranged, at the front in the direction of rotation, as protection against damage caused by weathering as a result of raindrops, dust, sand, particles of hail or as a result of flocks of birds. In modern offshore wind turbines, the diameters of the wind wheels are currently up to approximately 126 m, wherein the peripheral speed at the ends of the rotor blades reaches up to 500 km/h. If, at these speeds, rain or hail impacts the rotor, the rotor blades may be seriously damaged by the impact or the aerodynamic properties of the rotor and therefore the output of the wind turbine may be impaired by premature wear. Owing to the plastic composite component 10 with its elastomer layer 14 arranged beneath the thin, hard plastics outer layer 12, which preferably consists of UHMW-PE, the impact of such objects acting on the actual rotor blade 20 is much reduced so that damage is effectively prevented. The rotor blade 20 may also be protected in the other regions by such a plastic composite component 10 or may even be completely formed of such a plastic composite component. Alternatively, a design of the plastic composite component 10 as a cap is also possible, as is described for example in DE 10 2008 006 427 A1 on the basis of an erosion shield made of metal. The cap preferably covers the front outer fourth to fifth of the length of a rotor blade 20. This is the area in which there is the greatest risk of erosion or damage as a result of the high rotational speed. The hard, smooth surface of the plastics outer layer 12 also prevents corrosion and deposits on the rotor blade 20 as well as freezing, and therefore reduces the maintenance cost to a minimum. The slightly higher costs during production of the rotor blade 20 are offset many times over by lower maintenance costs and reduced downtime. The objective of maintenance-free operation of a wind turbine over a period of 20 years is achievable by the invention.

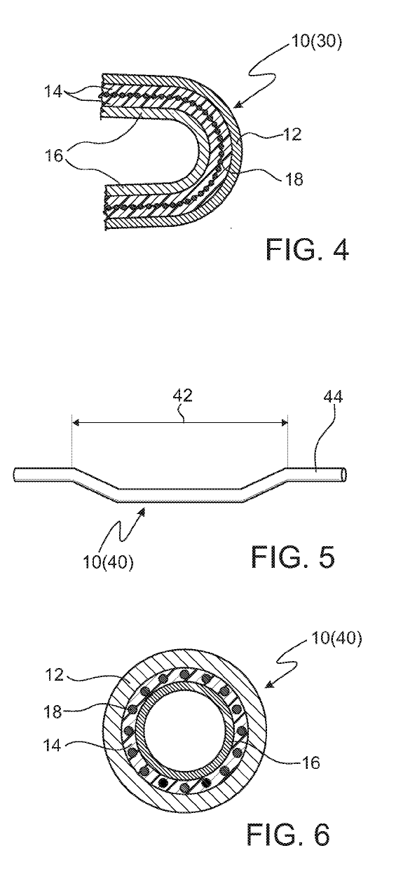

In FIG. 4 a plastic composite component 10 is formed as an edge protection composite component or a splinter protection composite component. An elastomer layer 14 and a plastics carrier layer 16 adjoin a plastics outer layer 12 on the inside. A woven fabric, a knitted fabric or a fiber structure 18 made of a high-performance fiber such as aramid or Vectran® (registered mark of Kuraray Co., Ltd., JP) is embedded in the elastomer layer 14 in such a way that the fibers are completely surrounded by the material of the elastomer layer 14, at least in the regions potentially exposed to an impact. The embedding preferably occurs in such a way that the fiber structure 18 is arranged within the elastomer layer 14 closer to the side of the plastic composite component 10 subject to tensile or bending tensile stresses, therefore in the present case closer to the plastics outer layer 12. The breaking strength and crack resistance of the fiber structure 18 is much increased by the embedding in the elastomer material. Tests have shown that such a plastic composite component 10 is able to withstand an impact energy, without damage to the component, which is more than 400% of the otherwise normal value. In the event of a crash, the risk of splintering or of further cracking of a broken component is drastically reduced.

The planar plastic composite component 10 illustrated in FIG. 1 and the edge protection composite component or splinter protection composite component 30 illustrated in FIG. 4 cover a large number of possible applications for components which are subjected to impact by a body. For example, in accordance with the invention these applications are:

the front edge of a wing, an airfoil or a tail unit of an aircraft, or

the front edge (in the direction of rotation) of a rotor blade of a helicopter or a wind wheel, or

a bodywork component of a vehicle, such as a bumper or a bonnet, or

a component of a vehicle subjected to swirling objects, such as an underbody protection part of a road vehicle or rail vehicle, a chassis strut, a steering gear, a driveshaft or cardan shaft, a pedal bearing or a chainstay of a mountain bike provided in particular with a carbon frame, or

an inner cladding of a land-, water-, air- or spacecraft, or

the inner face of a cavity of land-, water-, air- or spacecrafts, which cavity is accessible for maintenance purposes, for protection against damage caused by falling tools, or

surfaces facing the cargo of open or closed transport spaces, such as cargo holds of transporters and lorries or containers, or

the front hull region or the bilge region of a watercraft, such as a motorboat, a speedboat or a kayak, or

highly loaded effective areas of sports equipment, such as the blade area of ice hockey sticks, the base contact faces of Nordic walking sticks or ski sticks or the paddle blades of canoe or kayak paddles, or

a reinforcing part of an armor-plated vehicle

wherein the list above is understood to be merely exemplary and in no way exhaustive.

The plastic composite component or splinter protection composite component 10 illustrated in FIG. 4 also exhibits much improved crash behavior owing to the fiber structure 18 embedded in the elastomer layer 14, since in the event of a break less sharp-edged breaking edges and less loose breaking pieces are produced and the fibers ensure a residual stability. Examples of applications for this in accordance with the invention are as follows:

an inner cladding part of a vehicle, such as a door cladding, or

an outer bodywork component, such as a spoiler, a mudguard, a vehicle roof, a tailgate, a bonnet, a crash nose or a side part of a racing car, or

a frame, a handlebar, a handlebar front part or a seat pillar of a bicycle, a tennis racket, a rail or a heel cap of inline skates, a body protector or a protective helmet for protective clothing for work or leisure purposes (fire service, police, military and emergency services; bicycle, motorbike, skating or ski helmets or protectors)

wherein the list above is understood to be merely exemplary and in no way exhaustive.

A further application for a plastic composite component or splinter protection composite component 10 according to the invention is illustrated by way of example in FIGS. 5 and 6 on the basis of a bicycle handlebar 40. This application group concerns objects in which a total failure of the component is to be ruled out at any rate. For this purpose the bicycle handlebar 40 consists of a plastics outer layer 12, which is preferably formed of a carbon fiber structure (CRP). An elastomer layer 14 adjoins this on the inside, at least in the inner composite component region 42 of the bicycle handlebar 40, in which elastomer layer a woven fabric, a knitted fabric or a fiber structure 18, preferably made of a high-performance fiber such as aramid or Vectran® (registered mark of Kuraray Co., Ltd., JP) is embedded in such a way that the fibers are completely surrounded by the material of the elastomer layer 14. A further thin plastics carrier layer 16 optionally adjoins the elastomer layer 10 on the inside, which plastics carrier layer is also preferably formed of a carbon fiber structure (CRP). Should the plastics outer layer 12 of the bicycle handlebar 40 crack or break in the event of extreme loading (for example during downhill racing with a mountain bike), the bicycle handlebar 40 is still protected against sudden complete failure by the fiber structure 18 embedded in the elastomer layer 14. The rider can see the break forming in his handlebar in good time and accordingly reduce his speed, without falling off the bike since the bike can still be steered. In accordance with the invention, application objects from this group include, for example:

a rotor blade or an airfoil of a sporting plane, or

a prosthesis or orthesis, or

a frame, a handlebar, a handlebar front part or a seat pillar of a bicycle, a tennis racket, a rail or a heel cap of inline skates, a body protector or a protective helmet (motorbike helmet, bicycle helmet, occupational protective helmet or hat, fireman's helmet and the like), or

a front region (inlet region and first compression stage of an engine or a turbine which are exposed in particular to the risk of flocks of birds or, in the case of smaller engines on poor roads, to the risk of stone-chipping)

wherein the list above is understood to be merely exemplary and in no way exhaustive.

Such plastic composite components 10 with a fiber structure 18 embedded in an elastomer layer 14 are also very effectively integrated in protective clothing as protection against injury caused by shots, stabs and impacts in the field of personal protection (bullet-proof vests, protective shields) and in the case of corresponding types of sport (fencing, horse-riding, motorbike racing, motocross, ice hockey), or incorporated into corresponding protectors.

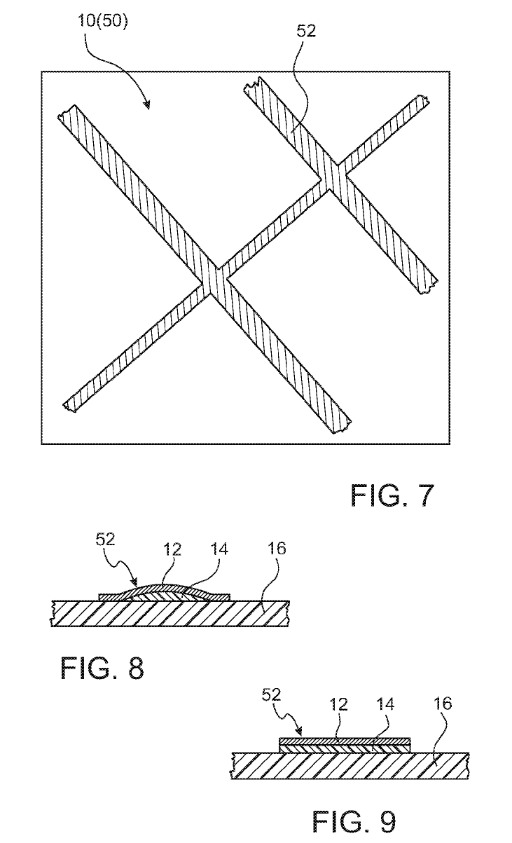

FIGS. 7 to 15 show a further group of inventive applications for plastic composite components 10. This group concerns components in which vibrations are to be damped and/or resonances are to be prevented or reduced and/or a reduction in acoustic vibrations is to be achieved. For this purpose at least one damping composite component 52 is provided by a planar plastic composite component 10, for example a bonnet 50, at least in partial areas, such as the strips indicated in FIG. 7. An elastomer layer 14 and, above this, a plastics outer layer 12 are arranged on a plastics carrier layer 16 in the partial areas of the damping composite component 52. The arrangement may also be reversed so that the layer denoted by reference numeral 16 in FIGS. 8 and 9 forms the plastics outer layer 12, adjoined on the inside by the elastomer layer 14 and the plastics carrier layer 16 instead of the layer 12. This possible reversal shows that, in principle, the plastics outer layer 12 may also function as a plastics carrier layer for all application objects described in this application, whilst the inner layer 16 has no bearing function in this instance, but merely limits the vibration-damping elastomer layer 14 on the inside. In FIG. 8 the elastomer layer 14 is completely covered by the layer 12, whereas in FIG. 9 the elastomer layer 14 is open to the sides.

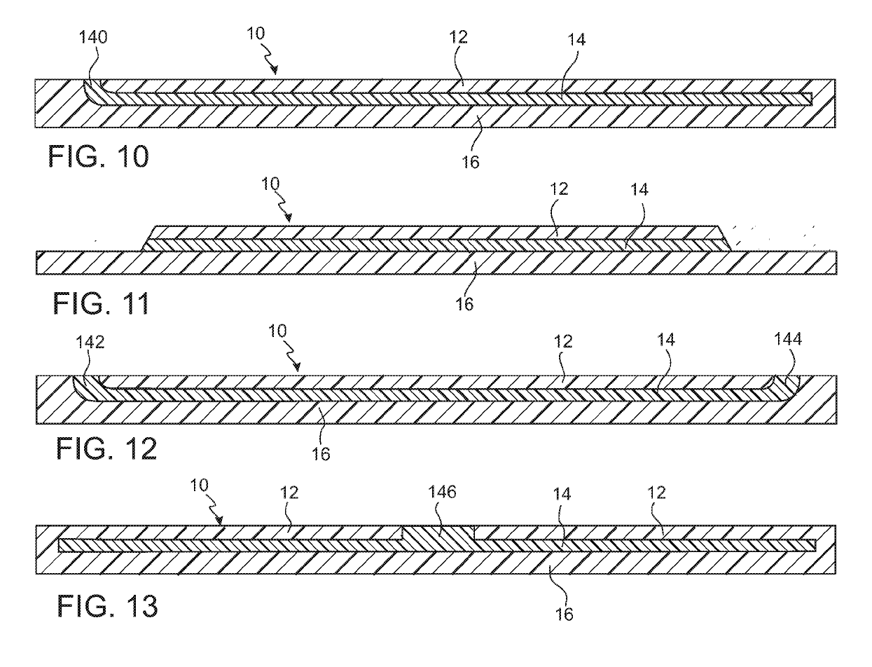

A number of embodiments for planar plastic composite components 10 are illustrated in FIGS. 10 to 13 which are able to effectively dampen vibrations, resonances and acoustic vibrations. In FIG. 10 an elastomer layer 14 is embedded between a plastics outer layer 12 and a plastics carrier layer 16 in such a way that it emerges at the plastics outer layer 12 by an exit region 140.

In FIG. 11 the elastomer layer 14 is placed on the plastics carrier layer 16 in a manner open on either side and is covered from above by a plastics outer layer 12.

In FIG. 12 the elastomer layer 14 is embedded between a plastics outer layer 12 and a plastics carrier layer 16 in such a way that it is in contact with the plastics outer layer 12 via two exit regions 142, 144.

In FIG. 13 the elastomer layer 14 is embedded between a plastics outer layer 12 and a plastics carrier layer 16 in such a way that it is connected to the surface of the plastics outer layer 12 via a central exit region 146.

The comments already made above for FIGS. 7 to 9 also apply to the embodiments according to FIGS. 10 to 13: the references to the plastics outer layer 12 and plastics carrier layer 16 can also be swapped in this instance so that a vibration damping on a component is also possible which is completely smooth on the outside. In this case the exit regions 140, 142, 144 and 146 are arranged on the inner face of the respective plastic composite component 10.

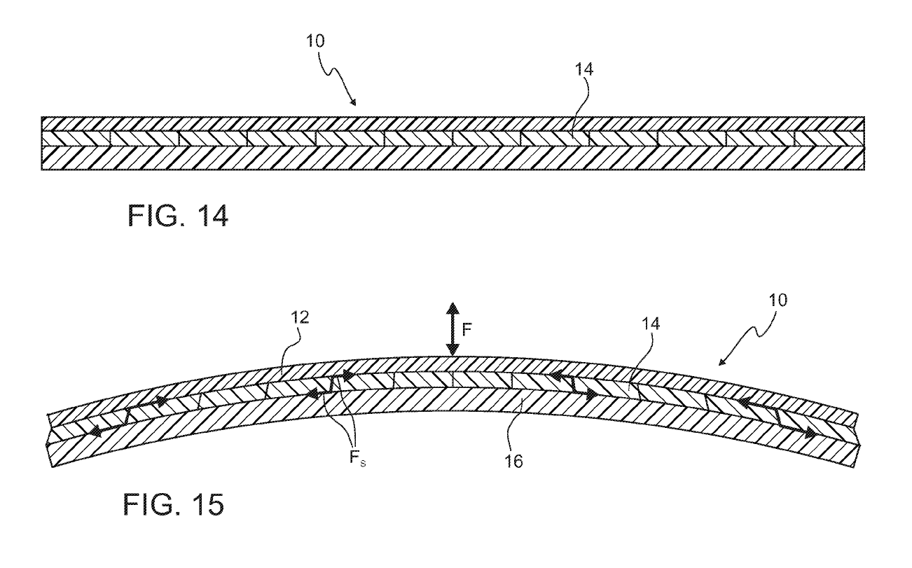

The manner in which the energy F of mechanical or acoustic vibrations is absorbed in the plastic composite component 10 is illustrated in FIGS. 14 and 15 by shear forces Fs arranged perpendicular thereto. The elastomer layer 14 absorbs the energy F of the vibrations at the boundaries with the plastics outer layer 12 and the plastics carrier layer 16 and converts this into shear forces Fs arranged perpendicular thereto. FIG. 14 shows the plastic composite component 10 in its idle position, whilst the plastic composite component 10 in FIG. 15 has been deflected in one direction by the force F of a vibration.

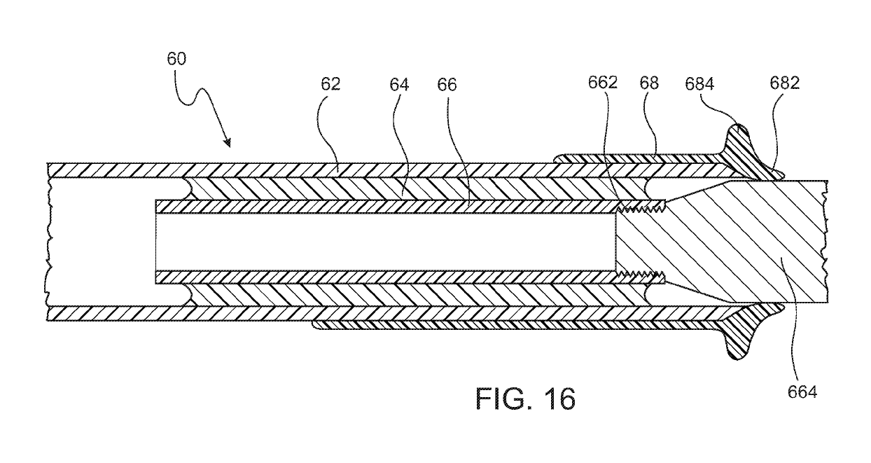

In FIG. 16 at least one grip of a shaft of a pneumatic hammer is formed as a vibration-damping plastic composite component 60. The tubular shaft of the pneumatic hammer with an outer diameter of approximately 50 mm comprises a plastics outer layer 62 which is formed of one of the materials indicated in claim 5, preferably of a fiber-reinforced composite plastic, in particular carbon fiber reinforced plastic (CRP). An elastomer or rubber layer 64 preferably adjoins the plastics outer layer 62 in the grip area to a length of approximately 15 cm, which elastomer or rubber layer is formed of one of the materials indicated in claims 6 to 11 or of a thermoplastic elastomer (TPE). The elastomer or rubber layer 64 may also be formed of a number of such layers. On the inside, the elastomer or rubber layer 64 adjoins a plastics carrier layer 66, the material of which preferably corresponds to that of the plastics outer layer 62. At one end the tubular plastics carrier layer 66 forms a tool support 662 which is formed as a thread in the embodiment shown and holds a tool 664. The thread is formed directly on the plastics carrier layer 66 in the embodiment shown. Instead of this, a metal thread piece may also be embedded in the plastics carrier layer 66. In contrast to the embodiment shown, the tool support 662 may also be formed as a bayonet fastener or as a conical support. A gripping region 68 made of a softer elastomer material is optionally additionally arranged on the plastics outer layer 62. This ensures a secure hold of the pneumatic hammer by the operator and an additional decoupling of vibrations. All layers 62, 64, 66 and 68 have preferably been bonded simultaneously in a single processing step in a manner to form the plastic composite component shown.

A sealing lip 682 is preferably molded on the gripping region 68 and protects the inside of the shaft containing the pneumatic components arranged therein against dirt and dust. A hand protection 684 in the form of an outwardly drawn lip which protects the user's hands against injury is also preferably molded on the end of the gripping region 68 facing the tool 664.

It is essential that the vibrations produced by the tool 664 in response to contact with the material to be machined (stones, concrete, asphalt, tiles) are largely decoupled or dampened by the elastomer layer 64 so that the user holding the gripping region is spared these vibrations as far as possible.

The same design illustrated in FIG. 16 is also suitable for a seat pillar of a bicycle. In this case, instead of the tool, the seat is fixed on the support 662, whilst the shaft comprising the plastics outer layer 62 is used for fastening on the frame tubing. The elastomer layer 64 decouples vibrations which would otherwise act on the seat as a result of unevennesses in the road.

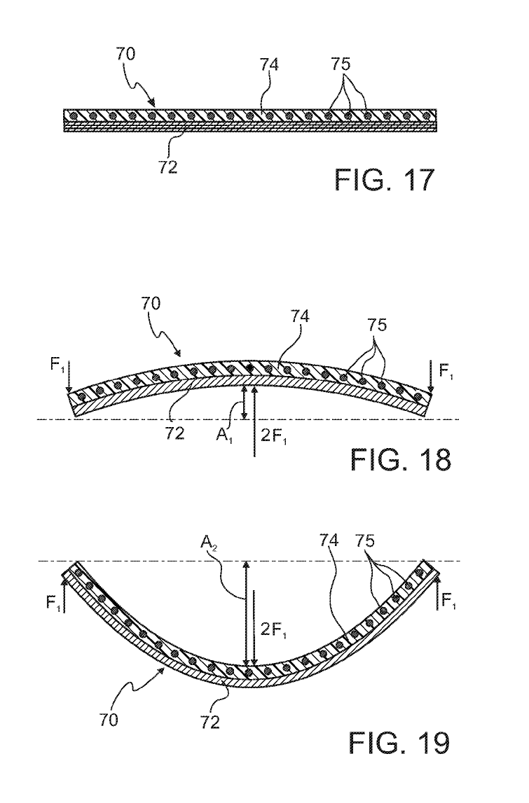

A plastic composite component 70 is shown in FIGS. 17 to 19 which is formed of a relatively thin, flexible plastics outer layer 72 and an elastomer layer 74 connected thereto with an embedded or incorporated woven fabric or fiber layer 75. The plastics outer layer 72 consists of one of the materials indicated in claim 5. It is preferably formed as a thin prepreg layer or SMC layer. The elastomer layer 74 is formed of one of the materials indicated in claims 6 to 11. The woven fabric, knitted fabric or fiber layer 75 embedded in the elastomer material of the elastomer layer 74 preferably consists of a high-performance fiber such as aramid or Vectran®. The plastic composite component 70 is flexible in both bending directions, wherein this leads to different deflections A1 and A2 with equal force. In FIG. 18 a force 2×F1 acting in the center of the component from the hard side of the plastics outer layer 72 which finds its restoring force per unit area in the lateral bearing forces F1 effects a deflection A1, which is much smaller than a deflection A2 which, according to FIG. 19, is produced by a force of equal size 2×F1 acting on the center of the plastic composite component 70 from the softer side of the elastomer layer 74. Such a different bending behavior provides completely new application possibilities, for example in leaf springs and in components loaded aerodynamically or hydrodynamically which deform considerably and “automatically” differently under the effect of pressure or base drag on different sides. For example, spoilers on high-speed vehicles, wings and airfoils of aircraft, propellers, turbines or the like are possible applications.

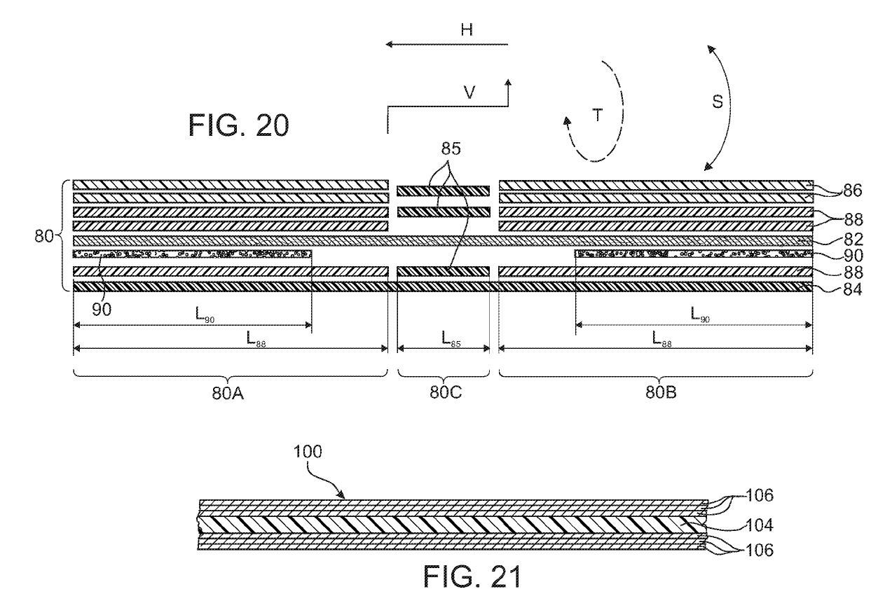

FIG. 20 shows a plastic composite component 80 which comprises a flexible articulation region 80C in the central area. The high-performance plastic composite component illustrated for use in motor racing comprises two layers of a carbon prepreg 86, therebelow two layers of a glass prepreg 88, therebelow a layer of a glass fiber fabric 82, therebelow an adhesive film 90 and therebelow an elastomer layer 84. The individual layers or coats are distanced vertically from one another in the illustration in FIG. 20 so as to be able to distinguish between them more clearly. In reality, all these layers form the plastic composite component 80 in an interconnected manner in which they are arranged closely on top of one another. Instead of the glass prepreg layers 88, further carbon prepreg layers 86 may also be provided. Whilst the glass fiber fabric 82 and the elastomer layer 84 pass through the articulation region in the embodiment shown, the layers of carbon prepreg 86 and glass prepreg 88 are interrupted in the articulation region 80C and, in this case, are bridged by elastomer portions 85 connected via their ends. In the central articulation region 80C a flexible connection of the rigid areas 80A and 80B of the plastic composite component 80 comprising the carbon prepreg layers 86 and the glass prepreg layers 88 and arranged to the right and left of said central articulation region is thus produced. In the embodiment, the articulation allows the following three degrees of freedom:

a pivoting or kinking movement S of the right-hand rigid part 80B relative to the left-hand rigid part 80B;

a vertical offset V of the right-hand rigid part 80B relative to the left-hand rigid part 80B, and lastly;

a horizontal displacement H of the right-hand rigid part 80B towards the left-hand rigid part 80B.

If the layer made of glass fiber fabric 82, which depending on requirements may also be replaced by a carbon fiber fabric, aramid fiber fabric or Vectran® fabric (registered mark of Kuraray Co., Ltd., JP), is interrupted in the articulation region 80C or replaced by a woven fabric made of resilient fibers, a limited horizontal displacement of the right-hand rigid part 80B away from the left-hand rigid part 80B is also additionally possible. Provided the articulation region 80C is only formed by one or more elastomer layers 84 and/or 85, a limited torsional movement T of the right-hand rigid part 80B relative to the left-hand rigid part 80B into and out of the plane of projection is also additionally possible. The strongest limitation of the movability of the articulation 80C is provided when at least one thin synthetic resin layer (carbon prepreg layer 86 or glass prepreg layer 88) spans the articulation region 80C. Only one limited pivoting movement S of the right-hand rigid part 80B relative to the left-hand rigid part 80B is then still possible. In this instance the fifth application of the plastic composite component 80 approximates the fourth application of the plastic composite component 70 according to FIGS. 17 to 19. The bond described there formed of a very thin, hard synthetic resin layer 72 and an elastomer layer 74 provided with a woven fabric or fiber layer 75 can also be used to produce a hinge-like articulation region on a plastic composite component 80. The possible fields of application are manifold:

door or flap hinges, for example for buildings, furniture or vehicles, but also for suitcases, chests, containers or other receptacles, wherein the tight design of the hinge area continuously along the hinge edge prevents the infiltration of air, liquid or particles in this area;

seals which have to be adapted to three-dimensional contours;

kinked areas of plates or hoses;

compensators for compensation of a vertical or lateral offset between two component faces;

aerodynamically advantageously formed transition by the avoidance of gaps between two components;

building claddings in corner regions (“soft edge protection” for example on pillars or quoins of underground parking areas),

articulation of movable flaps, such as landing flaps of aircraft, flow-directing flaps at retaining dams, rotor blades of helicopters;

flexible suspensions (for example in motor sport)

wherein the list above is understood to be merely exemplary and in no way exhaustive.

Plastic composite components 100 in which, according to FIG. 21, a core layer 104 made of an elastomer is arranged in the center, that is to say in the region of the neutral fibers of the plastic composite component 100, are advantageous for highly loaded components, for example bodywork parts of motorsport vehicles. A plurality of layers made of carbon fiber prepreg 106 (CRP) and/or glass fiber prepreg (GRP) and/or other fiber reinforced composite plastic layers (FRP), which form a smooth, hard surface, preferably adjoin the core layer 104 on the outside. In the embodiment three relatively thin carbon fiber prepreg layers 106 are provided on each side of the core layer 104. The core layer 104 makes the component considerably lighter compared to a pure CRP or GRP component, since the specific weight of the relatively thick elastomer layer is only approximately 1 g/cm3, whilst CRP has a specific weight of approximately 1.8 g/cm3 and GRP has a specific weight of approximately 2.0 g/cm3.

Compared to other known plastic composite components in which an extremely light core layer with a sandwich structure comprising spacers between two cover layers (honeycomb structure, for example made of cellulose or card) is used, the weight of the core layer 104 according to the invention made of an elastomer is higher; however the plastic composite component 100 according to the invention with the core layer 104 made of an elastomer affords considerable advantages compared to these extremely light composite components in terms of the impact behavior and vibration protection or damping behavior with regard to component vibrations. Owing to the integration of high-performance fibers into the neutral elastomer layer, a splinter protection can additionally be integrated.

The effective surfaces of a plastic composite component according to the invention can easily be adapted to the desired application, wherein in contrast to known composite components a connection of all layers is produced in a single processing step. The effective surface of the plastic composite material can be formed by the smooth, hard and scratch-resistant plastics outer layer, where minimal friction and good sliding properties (for example in the case of skis or snowboards), aerodynamic or hydrodynamic properties (for example in the case of airfoils or fuselages/hulls of air- or watercraft), protection against erosion, corrosion, abrasion and weathering (for example in the case of helicopter blades or wind wheels, in the case of external panels or external cladding parts of buildings or vehicles), or an avoidance of an adhesion of media or foreign bodies (for example in the case of containers of stirring devices, swimming pools or sewage treatment plant basins or in the case of ship hulls) are important.

By contrast, the effective surface of a plastic composite component according to the invention can be provided with a friction-increasing, soft layer made of an elastomer or TPE if said component requires a surface feel (for example gripping parts, steering wheels, switches and other operating elements) or anti-slip properties (for example surfaces of surfboards, internal cladding of freight holds, step plates in the entry and exit regions of vehicles).

A further field of application for a plastic composite component according to the invention is walls of fluid-guiding containers or pipelines. Owing to the embedded elastomer layer, such containers or tubes exhibit excellent protection against bursting. In particular in conjunction with a flame-resistant provision, such pipes and containers are best suited, for example, for the storage and guidance of chemically aggressive or highly explosive fluids. An additional coating formed of a rubber or rubber-like elastomer arranged on the medium-guiding side ensures the necessary media resistance of such containers or lines. Possible fields of application are fuel or oil tanks in all types of vehicle, in particular also in aircraft, helicopters or ships, also for military applications, pressurized air containers or lines, or tanks and lines for water, juices, other drinks, milk products or other foodstuffs, wherein a thermoplastic elastomer (TPE) is particularly well suited as a coating on the medium-guiding side.

The plastic composite component 10, 22, 42, 50, 60, 70, 80 or 100 with the plastics outer layer 12, 62, 72, 86 or 106 and the metal or plastics carrier layer 16, 66 or 88 as well as the elastomer layer 14, 64, 74; 84, 85 or 104 arranged there between is produced by being subjected to a treatment by way of an application of energy. For example, this may take place by a heat treatment in an oven, an autoclave, a heated press or a heated thermoforming die, a microwave system, a high-power light radiation system and/or a heatable table. The process temperature lies in the range of approximately 80 degrees Celsius to approximately 200 degrees Celsius, preferably at approximately 130 degrees Celsius. The duration of the process is approximately 5 hours. However, the duration of the process may vary within the given temperature range from approximately 10 minutes to approximately 8 hours depending on customer requirements. Alternatively, the plastic composite component 10 is subjected to another cross-linking treatment, for example with UV light. The at least one elastomer layer 14 cross-links with the synthetic resin of the plastics outer layer 12 and the plastics carrier layer 16. The plastics outer layer 12, the plastics carrier layer 16 and the elastomer layer or elastomer layers 12 are then bonded to one another in a non-detachable manner.

### LIST OF REFERENCE NUMERALS

10 plastic composite component
12 plastics outer layer
14 elastomer layer
140 exit region
142 exit region
144 exit region
146 exit region
16 plastics carrier layer
18 woven fabric/knitted fabric/fiber structure
20 rotor blade
22 plastic composite component (on 20)
30 edge protection composite component
40 bicycle handlebar
42 composite component region (of 40)
44 handlebar end (gripping region)
50 bonnet
52 damping composite component
60 plastic composite component
62 plastics outer layer
64 elastomer layer
66 plastics carrier layer
662 tool support
664 tool
68 gripping region
682 sealing lip
684 hand protection
70 plastic composite component
72 plastics outer layer
74 elastomer layer
75 woven fabric/knitted fabric/fiber layer
80 plastic composite component
82 glass fiber fabric
84 elastomer layer
85 elastomer portion
86 carbon prepreg
88 glass prepreg
90 adhesive film
100 plastic composite component
104 elastomer layer
106 carbon prepreg
F (vertical) vibration energy
Fs (horizontal) shear forces
F1 force
A1 (first) deflection
A2 (second) deflection
S pivoting movement
V vertical offset
H horizontal displacement
T torsion

Thus, it is seen that the objects of the present invention are efficiently obtained, although modifications and changes to the invention should be readily apparent to those having ordinary skill in the art, which modifications are intended to be within the spirit and scope of the invention as claimed. It also is understood that the foregoing description is illustrative of the present invention and should not be considered as limiting. Therefore, other embodiments of the present invention are possible without departing from the spirit and scope of the present invention.

What I claim is:

1. A synthetic material composite component, comprising the component formed of at least two layers, wherein a first layer formed of at least in part of a heat-curing synthetic resin and a second layer formed of at least in part of an elastomer are integrated by an energy input in a single processing procedure, and wherein the elastomer layer contains at least 0.5 pph (parts per hundred) of at least one cross-linking agent from the group of peroxides, amines and/or bisphenols, characterized in that, the synthetic material composite component is formed of a thin hard synthetic materials outer layer, at least one elastomer layer adjoining the former on the inside, and at least one metal and/or synthetic materials carrier layer, acting as a reinforcing support structure, adjoining said elastomer layer on the inside, and the synthetic materials carrier layer made of a fiber-reinforced synthetic material, and served as an impact protection part, or served as a splinter protection part or as a protection part against sudden total failure, or served as a protection part against vibrations and vibration damage, against resonances, wherein the synthetic materials carrier layer, which may optionally also be supplemented or replaced by at least one metal carrier layer, the elastomer layer positioned thereon and the synthetic materials outer layer are jointly assembled in a single processing procedure and are then jointly cured or vulcanized under the effect of temperature in Autoclave or Hot press.

2. The synthetic material composite component according to claim 1, characterized in that the application of energy is a joint heat treatment or a radiation with UV light; the fiber-reinforced synthetic material is a carbon fiber reinforced synthetic material or a glass fiber reinforced synthetic material.

3. The synthetic material composite component according to claim 1, characterized in that the synthetic material composite component is arranged as an impact protection part on the front edge of a wing, an airfoil or a tail unit of an aircraft, or on the front edge in the direction of rotation of a rotor blade of a helicopter or a wind wheel, or on a bodywork component of a vehicle, or on a component of a vehicle subjected to swirling objects, or on an inner cladding of a land, water, air or spacecraft, or on the inner face of a cavity of land, water, air or spacecrafts, which cavity is accessible for maintenance purposes, for protection against damage caused by falling tools, or on the surfaces facing the cargo of open or closed transport spaces, or on the front hull region or the bilge region of a watercraft, or on highly loaded effective areas of sports equipment or at least partly forms the latter.

4. The synthetic material composite component according to claim 3, characterized in that the bodywork component of a vehicle is a bumper or a bonnet; the component of a vehicle is an underbody protection part of a road vehicle or rail vehicle, a chassis strut, a steering gear, a driveshaft or cardan shaft; the transport spaces is cargo holds of transporters or containers; the watercraft is a motorboat, a speedboat or a kayak; the highly loaded effective areas of sports equipment is the blade area of ice hockey sticks, the base contact faces of Nordic walking sticks or ski sticks or the paddle blades of canoe or kayak paddles.

5. The synthetic material composite component according to claim 3, characterized in that the component of a vehicle is a bearing or chain support of a mountain bike provided with a carbon frame; the transport spaces is cargo holds of lorries.

6. The synthetic material composite component according to claim 1, characterized in that the synthetic material composite component is arranged as a splinter protection part or as a protection part against sudden total failure on an inner cladding part of a vehicle, or on an outer bodywork component, or on a rotor blade, an airfoil of a sporting plane, or on a prosthesis or orthesis, or on a frame, a handlebar, a handlebar front part or a seat pillar of a bicycle, a tennis racket, a rail or a heel cap of inline skates, a body protector, a protective helmet, or on a reinforcing part of an armor-plated vehicle, or on a strengthening part of an armored vehicle, or on a splinter protection part or stabbing protection part as protection against injury caused by shots, stabs or impacts or at least partly forms the latter.

7. The synthetic material composite component according to claim 6, characterized in that the inner cladding part of a vehicle is a door cladding; the outer bodywork component is a spoiler, a mudguard, a vehicle roof, a tailgate, a bonnet, a crash nose or a side part of a racing car.

8. The synthetic material composite component according to claim 1, characterized in that the synthetic material composite component is arranged as a protection part against vibrations and vibration damage, against resonances, for vibration damping or for acoustic damping on an inner cladding part of a vehicle, or on an outer bodywork component of a vehicle, or on components subjected to vibrations in machine construction, or on turbines, rotor blades or wings, or on measuring devices or on optical devices, or on construction machinery, or on slider or snowboards, or on bridges or at least partly forms the latter.

9. The synthetic material composite component according to claim 8, characterized in that the inner cladding part of a vehicle is a door cladding; the outer bodywork component is a bumper, a vehicle roof, a vehicle door, a boot lid or a bonnet; the components subjected to vibrations is a robot arm of an industrial robot, a gripping area of a pneumatic tool, a frame, mount or stand.

10. The synthetic material composite component according to claim 1, characterized in that the synthetic materials outer layer is formed of a woven fabric material which is already saturated with synthetic resin or prepreg or is produced by the resin infusion method.

11. The synthetic material composite component according to claim 1, characterized in that the synthetic materials outer layer is formed of a fiber-reinforced composite synthetic material, or of polyethylene or polytetrafluoroethylene.

12. The synthetic material composite component according to claim 11, characterized in that the fiber-reinforced synthetic material is a carbon fiber reinforced synthetic material or a glass fiber reinforced synthetic material; the polyethylene is ultra high molecular weight polyethylene or high molecular weight polyethylene.

13. The synthetic material composite component according to claim 1, characterized in that the at least one elastomer layer consists of ethylene-propylene rubber, ethylene-propylene-diene rubber, ethylene-acrylate rubber, fluorocarbon rubber, acrylate rubber, acrylonitrile-butadiene rubber, optionally mixed with polyvinyl chloride, hydrogenated nitrile rubber, carboxylate-nitrile rubber, hydrogenated carboxylate-nitrile rubber, natural rubber, ethyl vinyl acetate, chlorosulfonyl-polyethylene rubber, chlorinated polyethylene, butyl rubber or halobutyl rubber, silicone rubber, fluorosilicone rubber, chlorohydrin rubber, epichlorohydrin rubber, polychloroprene rubber, one-component polyurethane or a combination or a offcut of the aforementioned substances, wherein 0.5 to 15 pph of a peroxide are provided as a cross-linking agent.

14. The synthetic material composite component according to claim 13, characterized in that the silicone rubber is VMQ or MVQ, the fluorosilicone rubber is FVMQ or MFQ; wherein 1.5 to 5 pph of a peroxide are provided as a cross-linking agent.

15. The synthetic material composite component according to claim 1, characterized in that the at least one elastomer layer consists of ethylene-propylene rubber, ethylene-propylene-diene rubber, ethylene-acrylate rubber, fluorocarbon rubber, acrylate rubber, acrylonitrile-butadiene rubber, optionally mixed with polyvinyl chloride, hydrogenated nitrile rubber, carboxylate-nitrile rubber, hydrogenated carboxylate-nitrile rubber, natural rubber, ethyl vinyl acetate, chlorosulfonyl-polyethylene rubber, silicone rubber, fluorosilicone rubber, one-component polyurethane or a combination or a offcut of the aforementioned substances, wherein 0.5 to 25 pph of an acrylate-based heat-curing resin are additionally provided to assist the cross-linking agent.

16. The synthetic material composite component according to claim 15, characterized in that the silicone rubber is VMQ or MVQ, the fluorosilicone rubber is FVMQ or MFQ; wherein 1.5 to 5 pph of an acrylate-based heat-curing resin are additionally provided to assist the cross-linking agent.

17. The synthetic material composite component according to claim 1, characterized in that the at least one elastomer layer consists of ethylene-acrylate rubber, fluorocarbon rubber, acrylate rubber, ethyl vinyl acetate, chlorosulfonyl-polyethylene rubber, chlorinated polyethylene, chlorohydrin rubber, epichlorohydrin rubber, polychloroprene rubber or a combination or a offcut of the aforementioned substances and 0.5 to 15 pph of an amine are provided as a cross-linking agent.

18. The synthetic material composite component according to claim 17, characterized in that 1.5 to 5 pph of an amine are provided as a cross-linking agent.

19. The synthetic material composite component according to claim 1, characterized in that the at least one elastomer layer consists of fluorocarbon rubber and 0.5 to 15 pph of a bisphenol are provided as a cross-linking agent.

20. The synthetic material composite component according to claim 19, characterized in that 1.5 to 5 pph of a bisphenol are provided as a cross-linking agent.

21. The synthetic material composite component according to claim 1, characterized in that the at least one elastomer layer consists of chlorohydrin rubber, epichlorohydrin rubber, polychloroprene rubber or a combination or a offcut blend of the aforementioned substances and 0.5 to 15 pph of a thiourea, a thiourea derivative or a dithiocarbonate derivative are provided as a cross-linking agent.

22. The synthetic material composite component according to claim 21, characterized in that 1.5 to 5 pph of a thiourea, a thiourea derivative or a dithiocarbonate derivative are provided as a cross-linking agent.

23. The synthetic material composite component according to claim 1, characterized in that the at least one elastomer layer consists of ethylene-propylene rubber, ethylene-propylene-diene rubber, ethylene-acrylate rubber, fluorocarbon rubber, acrylate rubber, acrylonitrile-butadiene rubber, optionally mixed with polyvinyl chloride, hydrogenated nitrile rubber, carboxylate-nitrile rubber, hydrogenated carboxylate-nitrile rubber, natural rubber, ethyl vinyl acetate, chlorosulfonyl-polyethylene rubber, silicone rubber, one-component polyurethane or a combination or a offcut of the aforementioned substances and 0.5 to 15 pph of a phenol resin are additionally provided to assist the cross-linking agent.

24. The synthetic material composite component according to claim 23, characterized in that the silicone rubber is VMQ or MVQ; wherein 1.5 to 5 pph of a phenol resin are additionally provided to assist the cross-linking agent.

25. The synthetic material composite component according to claim 1, characterized in that the at least one first layer made of a thermosetting synthetic material and is provided with a fiber insert, at least one second layer consists of a thermosynthetic material elastomer.

26. The synthetic material composite component according to claim 1, characterized in that the thermosynthetic material elastomer is styrene/ethylene-butene/styrene block copolymer or styrene/butadiene/styrene block copolymer or a thermosynthetic material elastomer based on polyurethane or a low-density polyethylene or styrene/butadiene rubber with a styrene content of more than 50%.

27. The synthetic material composite component according to claim 1, characterized in that the at least one elastomer layer consists of a thermosynthetic material elastomer, which is styrene/ethylene-butene/styrene block copolymer or styrene/butadiene/styrene block copolymer or a thermosynthetic material elastomer based on polyurethane or a low-density polyethylene or styrene/butadiene rubber with a styrene content of more than 50%.

28. The synthetic material composite component according to claim 1, characterized in that at least one woven fabric or a knitted fabric or a fiber structure is embedded in the at least one elastomer layer in such a way that the fibers of said woven fabric or knitted fabric or fiber structure are surrounded completely by the elastomer, at least in partial regions of the area.

29. The synthetic material composite component according to claim 28, characterized in that the woven fabric or the knitted fabric or the fiber structure is embedded in the elastomer layer in such a way that it is arranged closer to the side of a tensile load or bending tensile load of the synthetic material composite component.

30. The synthetic material composite component according to claim 28, characterized in that the woven fabric or the knitted fabric or the fiber structure consists of a high-performance fiber.

31. The synthetic material composite component according to claim 30, characterized in that the high-performance fiber is aramid or Vectran®.

32. The synthetic material composite component according to claim 1, characterized in that the elastomer layer is arranged as a core layer in the region of the neutral fibers of the synthetic material composite component.

33. The synthetic material composite component according to claim 32, characterized in that the elastomer layer arranged in the centre of the synthetic material composite component as a core layer is connected on both sides by a plurality of layers of a carbon prepreg.

34. The synthetic material composite component according to claim 1, characterized in that it is used as a wall of a media-guiding container or a media-guiding line which is provided on its side facing the medium with an additional coating made of an elastomer.
